SEQUENCE LISTING (2 sequences)

SEQ ID NO: 1 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - DNA, 1692 bp, Homo sapiens
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctga

(19) United States
(12) Patent Application Publication
(10) Pub. No.: US 2020/0360491 A1
(43) Pub. Date: Nov. 19, 2020

(54) TREATMENT OF LYSOSOMAL STORAGE DISEASE IN THE EYE THROUGH ADMINISTRATION OF AAVS EXPRESSING TPP1

(71) Applicant: THE CHILDREN'S HOSPITAL OF PHILADELPHIA, Philadelphia, PA (US)

(72) Inventors: Beverly DAVIDSON, Philadelphia, PA (US); Luis TECEDOR, Philadelphia, PA (US)

(73) Assignee: THE CHILDREN'S HOSPITAL OF PHILADELPHIA, Philadelphia, PA (US)

(21) Appl. No.: 16/843,360

(22) Filed: Apr. 8, 2020

Related U.S. Application Data
(60) Provisional application No. 62/831,067, filed on Apr. 8, 2019.

(51) Int. Cl. A61K 38/48; A61K 35/761; A61K 38/17; A61P 27/02

(52) U.S. Cl. CPC A61K 38/48; A61P 27/02; A61K 38/1719; A61K 35/761

(57) ABSTRACT

Provided are methods of treating the retinal dysfunction in a mammal with lysosomal storage disorder which method comprises sub-retinal administration of recombinant AAV particles encoding a soluble lysosomal tripeptidyl peptidase 1 (TPP1). In particular, the retinal dysfunction may be occurring in children with CLN2 deficiency receiving enzyme replacement therapy or gene therapy for their disease.

### REFERENCE TO RELATED APPLICATIONS

The present application claims the priority benefit of U.S. provisional application No. 62/831,067, filed Apr. 8, 2019, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 8, 2020, is named CHOPP0026US_ST25.txt and is 7.6 kilobytes in size.

### BACKGROUND

### 1. Field

The present disclosure relates to the fields of medicine, genetics, and molecular biology. More specifically, it deals with the subretinal administration of AAV vectors expressing the lysosomal serine protease TPP1 for the treatment of lysosomal storage disease.

### 2. Related Art

Gene transfer is now widely recognized as a powerful tool for analysis of biological events and disease processes at both the cellular and molecular level. More recently, the application of gene therapy for the treatment of human diseases, either inherited (e.g., ADA deficiency) or acquired (e.g., cancer or infectious disease), has received considerable attention.

Traditionally, gene therapy has been defined as a procedure in which a therapeutic gene is introduced into cells of a mammal in order to correct an inborn genetic error. Although more than 4500 human diseases are currently classified as genetic, specific mutations in the human genome have been identified for relatively few of these diseases. Until recently, these rare genetic diseases represented the exclusive targets of gene therapy efforts. Accordingly, most of the NIH approved gene therapy protocols to date have been directed toward the introduction of a functional copy of a defective gene into the somatic cells of an individual having a known inborn genetic error. Only recently, have researchers and clinicians begun to appreciate that most human cancers, certain forms of cardiovascular disease, and many degenerative diseases also have important genetic components, and for the purposes of designing novel gene therapies, should be considered “genetic disorders.” Therefore, gene therapy has more recently been broadly defined as the correction of a disease phenotype through the introduction of new genetic information into the affected organism.

In in vivo gene therapy, a transferred gene is introduced into cells of the recipient organism in situ that is, within the recipient. In vivo gene therapy has been examined in several animal models. Several recent publications have reported the feasibility of direct gene transfer in situ into organs and tissues such as muscle, hematopoietic stem cells, the arterial wall, the nervous system, and lung. Direct injection of DNA into skeletal muscle, heart muscle and injection of DNA-lipid complexes into the vasculature also has been reported to yield a detectable expression level of the inserted gene product(s) in vivo.

Treatment of diseases of the central nervous system, e.g., inherited genetic diseases of the brain, remains an intractable problem. Examples of such are the lysosomal storage diseases and Alzheimer's disease. Collectively, the incidence of lysosomal storage diseases (LSD) is 1 in 10,000 births worldwide, and in 65% of cases, there is significant central nervous system (CNS) involvement. Proteins deficient in these disorders, when delivered intravenously, do not cross the blood-brain barrier, or, when delivered directly to the brain, are not widely distributed. Thus, therapies for the CNS deficits need to be developed.

### SUMMARY

Thus, in accordance with the present disclosure, there is provided a method of treating a mammal having a lysosomal storage disease (LSD), said method comprising sub-retinal administering to a mammal in need thereof a plurality of AAV particles, said AAV particles comprising (i) a nucleic acid inserted between a pair of AAV inverted terminal repeats (ITRs), said nucleic acid encoding (1) a soluble lysosomal tripeptidyl peptidase 1 (TPP1) polypeptide, (2) a fragment thereof, (3) a proenzyme of either of the foregoing, or (4) a combination of any of the foregoing; and (ii) an expression control element operably linked to and driving expression of said nucleic acid to yield a polypeptide having lysosomal hydrolase activity, wherein said AAV particles are capable of transducing cells of said mammal and providing expression of said polypeptide. The one or more of the AAV ITRs may comprise one or more AAV2 ITRs. The nucleic acid may encode mammalian TPP1, such as human TPP1. The method may result in a slowing, stopping, reversing, or preventing vision loss/blindness.

The expression control element may comprise a CMV enhancer and/or a beta actin promoter, such as a chicken beta actin promoter. The expression control element comprises a sequence having 80% or more identity to a native CMV enhancer or 80% or more identity to a native chicken beta actin promoter or to functional fragments of any of the foregoing.

The AAV particles further comprise a capsid protein. The capsid sequence or fragment may comprise a VP1, VP2 and/or VP3 capsid sequence or fragment having 70% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences or functional fragments thereof. The capsid sequence or fragment may comprise a VP1 capsid sequence or fragment having 80% or more identity to AAV2, wherein the capsid sequence or fragment has a tyrosine at positions 444, 500 and/or 730 substituted with an amino acid that is not tyrosine. The capsid sequence or fragment may comprise a VP1 capsid sequence or fragment having 90% or more identity to AAV2 or functional fragments thereof, wherein the capsid sequence or fragment has a tyrosine at positions 444, 500 and/or 730 substituted with phenylalanine. The capsid sequence or fragment may comprise an AAV2 VP1 capsid sequence or functional fragments thereof having a tyrosine at positions 444, 500 and/or 730 substituted with phenylalanine. The capsid sequence or fragment may comprise a VP1, VP2 or VP3 capsid sequence or functional fragments thereof selected from any of: AAV1, AAV2, AAV3, AAV4, AAV5, AAV6, AAV7, AAV8, AAV9, AAV10, AAV11, Rh10, Rh74 or AAV-2i8 AAV serotypes or functional fragments thereof.

The patient may have previously, currently or will receive TPP1 enzyme replacement therapy through a distinct route of administration. The AAV particles may be administered at a dose of about 1×10⁸ to about 1×10¹⁵ total vg. The mammal may be a non-rodent mammal, such as a primate, such as a human, such as a human child, such as human child from about 1 to about 4 years of age. The LSD may be infantile or late infantile ceroid lipofuscinoses (LINCL), Juvenile Batten, Fabry, MLD, Sanfilippo A, Krabbe, Morquio, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GMI Gangliosidosis, Mucolipidosis type II7ΠI, or Sandhoff disease. Patients with comorbidities of one or more of these diseases is also contemplated. The onset of a symptom associated with said LSD may be delayed by 5-10, 10-25, 25-50 or 50-100 days. The symptom may be selected from the group consisting of proionceptive response, nystagmus, menace, pupillary light reflex, cerebellar ataxia, intention tremor, or any combination of any of the foregoing. The symptom may be vision loss/blindness.

The AAV particles may be selected from the group consisting of AAV1, AAV2, AAV3, AAV4, AAV5, AAV6, AAV7, AAV8, AAV9, AAV10, AAV11, AAV 12, AAV-rh74, AAV-Rh10 and AAV-2i8 particles or functional fragments thereof. The one or more of said ITRs may be selected from the group consisting of an AAV1, AAV2, AAV3, AAV4, AAV5, AAV6, AAV7, AAV8, AAV9, AAV10, AAV11, AAV 12, AAV-rh74, AAV-Rh10 and AAV-2i8 ITR. The capsid sequence or fragment may comprise a VP1, VP2 and/or VP3 capsid sequence or functional fragments thereof having 90% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences or functional fragments thereof. The capsid sequence or fragment may comprise a VP1, VP2 or VP3 capsid sequence selected from any of: AAV1, AAV2, AAV3, AAV4, AAV5, AAV6, AAV7, AAV8, AAV9, AAV10, AAV11, Rh1O, Rh74 or AAV-2i8 AAV serotypes.

Other objects, features, and advantages of the present disclosure will become apparent from the following detailed description. It should be understood, however, that the detailed description and the specific examples, while indicating particular embodiments of the disclosure, are given by way of illustration only, since various changes and modifications within the spirit and scope of the disclosure will become apparent to those skilled in the art from this detailed description.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

Embodiments of the invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F. TPP1 levels in non-human primate eyes after AAV-TPP1 subretinal injection. (FIGS. 1A-B) TPP1 levels in retina at experimental endpoint (8 weeks post-injection) after different AAV-TPP1 doses. Vehicle (open bars), doses of 7.5e11, 2.5e11, 8.3e10, 8.3e9, 5.0e9 total vgs. FIG. 1A is 8 weeks post-injection; FIG. 1B is 22 weeks post-injection. TPP1 levels in each sample were normalized with the amount of retinal tissue using RPE65, a specific retinal pigment epithelium marker as a normalizer. Both a linear scale and log scale are provided for each. (FIGS. 1C-D) TPP1 levels in aqueous humor along the time after AAV-TPP1 injection. Increased TPP1 concentration reached plateau expression in aqueous humor between 4 to 7 weeks after AAV-TTP1 injection. (FIG. 1C) Doses of 7.5e11, 2.5e11, 8.3e10 total vgs. (FIG. 1D) Doses of 8.3e9, 5.0e9 total vgs. Both a linear scale and a log scale are provided for each. (FIG. 1E) No changes in TPP1 levels respective to endogenous expression in optic nerve at this early time point (22 weeks after treatment). Vehicle injected (open bars), 5.0e9, and 8.3e9 total vgs (black bars) groups. TPP1 levels were normalized to GAPDH. (FIG. 1F) Percentage of TPP1 activity after incubation of TPP1-deficient cells with recombinant TPP1 and serum from control or injected animals. Decreased TPP1 activity can be quantified in samples from animals which developed neutralizing antibodies against the recombinant TPP1. (FIGS. 1A,B,E,F) Injected (black bars) and uninjected eyes (open bars).

FIG. 2. Protein (SEQ ID NO: 1) and DNA (SEQ ID NO: 2) sequences for TPP1.

### DETAILED DESCRIPTION

As discuss above, treatment of diseases of the central nervous system, e.g., inherited genetic diseases of the brain, remains an intractable problem. Proteins deficient in these disorders, when delivered intravenously, do not cross the blood-brain barrier, or, when delivered directly to the brain, are not widely distributed. Neuronal Ceroid Lipofuscinoses (NCLs) is a group of childhood neurodegenerative diseases also known as Batten's Disease. CLN2 disease, an inherited autosomal recessive disorder, is one of the more common forms of NCL. Mutations in the TPP1 gene cause a deficiency of the soluble lysosomal enzyme tripeptidyl peptidase 1 (TPP1) resulting in an accumulation of lysosomal waste in brain and eye cells leading to the development of CLN2 disease, characterized by seizure disorder, developmental delay, and progressive blindness. Enzyme replacement therapy, designed to restore TPP1 enzyme activity and reduce the symptoms of CLN2 disease, has improved the quality of life of children with the disease. It is presumed that this treatment will also extend the lifespan of these children. However, the recombinant protein, which is delivered to the brain every two weeks via an infusion into the lateral cerebellar ventricle via an omaya reservoir, cannot correct the progressive vision loss.

The inventors determined whether subretinal delivery of AAV2-TPP1 could restore TPP1 enzyme activity in the eyes of these patients and prevent the ensuing loss of sight. They developed a novel method of subretinal administration of a gene therapy vector, rAAV particle comprising an AAV capsid protein and a vector comprising a nucleic acid encoding the TPP1 gene inserted between a pair of AAV inverted terminal repeats to correct the lack of endogenous TPP1 activity in the eye. In biodistribution and dose NHP studies, the inventors were successful in demonstrating that subretinal injection of the test particle, AAV2-TPP1, expressed human TPP1 protein and sustained lysosomal TPP1 enzymatic activity for the duration of an 8-week study. Endogenously expressed TPP1 is synthesized as a catalytically inactive enzyme, auto-catalytically processed into a mature active enzyme after entry into the acidic environment of lysosomes. The expressed TPP1 corrects both the transduced cells and neighboring cells by virtue of the fact that some TPP1 is secreted and is taken up by non-transduced cells. Because TPP1 can be secreted and used to cross-correct non-transduced cells of the retina, this gene therapy approach provides a method for treating the entire thickness of the retina after a one-time delivery to the subretinal space. These and other aspects of the disclosure are set out in greater detail below.

### I. Lysosomal Storage Diseases

Provided herein are methods and uses for administering to a mammal, in need of a method described herein, that is suspected of having or that has a lysosomal storage disease (LSD). In certain embodiments, a method or use described herein is used to treat, prevent, inhibit, reduce, decrease or delay the number, severity, frequency, progression or onset of one or more symptoms of an LSD.

Non-limiting examples of LSDs include Infantile Lipofuscinosis or Late infantile Neuronal Ceroid Lipofuscinosis (LINCL), Gaucher, Juvenile Batten, Fabry, MLD, Sanfilippo A, Late Infantile Batten, Hunter, Krabbe, Morquio, Pompe, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type 117111, or Sandhoff disease.

LSDs are often caused by a genetic abnormality (e.g., mutation, deletion, insertion) in the gene encoding a tripeptidyl peptidase-1 (TPP1) enzyme thereby leading to a deficiency of functional TPP1 enzyme activity. In humans, TPP1 is encoded by the CLN2 gene, sometimes called the TPP1 gene. For example, late infantile Neuronal Ceroid Lipofuscinosis (LINCL) is a childhood neurodegenerative disease caused most often by deficiency of TPP1 activity, due to mutations in CLN2. Development is normal up to ages 2-4 years after which manifestations of LINCL present as motor and mental decline, seizure disorder and visual deficits. Death generally occurs within the first decade of life. Most cases of LINCL are due to mutations in CLN2, which induce a deficiency of the soluble lysosomal enzyme tripeptidyl peptidase-1 (TPP1). TPP1 is synthetized as a mannose-6-phophate pro-enzyme and, similar to other soluble lysosomal hydrolases, the pro-enzyme is largely targeted to the lysosome but can also be released from the cell via the secretory pathway. As such, cellular uptake by the same or neighboring cells, and subsequent lysosomal delivery and activation of the proenzyme to the active form, can occur.

In certain embodiments, provided herein are methods of treating a mammal having, or suspected of having an LSD by administering, directly to a tissue or fluid of the central nervous system, AAV particles that direct the expression of polypeptide having TPP1 activity (referred to herein as AAV-TPP1 particles). Disclosed herein are data showing AAV delivery/administration to the brain and/or spinal cord in an animal model of a lysosomal storage disorder.

Any suitable mammal can be treated by a method or use described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models used for the study of LSDs, such as LINCL.

Subjects treated by a method or composition described herein include adults (18 years or older) and children (less than 18 years of age). Children range in age from 1-2 years old, or from 2-4, 4-6, 6-18, 8-10, 10-12, 12-15 and 15-18 years old. Children also include infants. Infants typically range from 1-12 months of age.

### II. AAV Vectors

Adeno associated virus (AAV) is a small nonpathogenic virus of the parvoviridae family. To date, numerous serologically distinct AAVs have been identified, and more than a dozen have been isolated from humans or primates. AAV is distinct from the other members of this family by its dependence upon a helper virus for replication.

AAV genomes been shown to stably integrate into host cellular genomes; possess a broad host range; transduce both dividing and non-dividing cells in vitro and in vivo and maintain high levels of expression of the transduced genes. AAV viral particles are heat stable, resistant to solvents, detergents, changes in pH, temperature, and can be concentrated on CsCl gradients or by other means. The AAV genome comprises a single-stranded deoxyribonucleic acid (ssDNA), either positive- or negative-sensed. In the absence of a helper virus, AAV may integrate in a locus specific manner, for example into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome. In cases where recombinant plasmids are used to construct or manufacture recombinant vectors, the vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant virus production but is not itself packaged or encapsulated into virus (e.g., AAV) particles. Thus, a vector “genome” refers to nucleic acid that is packaged or encapsulated by virus (e.g., AAV).

The AAV virion (particle) is a non-enveloped, icosahedral particle approximately 25 nm in diameter. The AAV particle comprises a capsid of icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single- stranded progeny DNA or infectious particles. An AAV particle is a viral particle comprising an AAV capsid or fragment. In certain embodiments the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the nonstructural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments, one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank its single stranded viral DNA genome. The ITR sequences often comprise about 145 bases each. Within the ITR region, two elements have been described which are thought to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments an AAV particle (e.g., an rAAV) comprises two ITRs. In certain embodiments an AAV (e.g., an rAAV) comprises a pair of ITRs. In certain embodiments an AAV particle (e.g., an rAAV) comprises a pair of ITRs that flank (i.e., are at each 5′ and 3′ end) of a polynucleotide that at least encodes a polypeptide having TPP1 enzyme activity.

A viral vector is derived from or based upon one or more nucleic acid elements that comprise a viral genome. Particular viral vectors include adeno-associated virus (AAV) vectors. Also provided are vectors (e.g., AAV) comprising a nucleic acid sequence encoding a TPP1 polypeptide, variant or subsequence (e.g., a polypeptide fragment having TPP1 enzyme activity).

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a nucleic acid (e.g., gene) encoding a TPP1 polypeptide is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a TPP1 encoding nucleic acid sequence, to add non-native nucleic acid such as a TPP1 encoding nucleic acid sequence, or a combination thereof. Typically, for AAV one or both inverted terminal repeat (ITR) sequences of AAV genome are retained in the AAV vector. A “recombinant” viral vector (e.g., rAAV) is distinguished from a viral (e.g., AAV) genome, since all or a part of the viral genome has been replaced with a non-native sequence with respect to the viral (e.g., AAV) genomic nucleic acid such as TPP1 encoding nucleic acid sequence, non-native nucleic acid such as a TPP1 encoding nucleic acid sequence has been added, or a combination thereof. Incorporation of a non-native sequence therefore defines the viral vector (e.g., AAV) as a “recombinant” vector, which in the case of AAV can be referred to as a “rAAV vector.”

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is an rAAV particle. A rAAV particle often comprises an AAV vector, or a portion thereof. A rAAV particle can be one or more AAV particles (e.g., a plurality of AAV particles). rAAV particles typically comprise proteins that encapsulate or package the rAAV vector genome (e.g., capsid proteins).

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV 12, AAV-rh74, AAV-rhlO or AAV-2i8. In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross -reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome is identical to the serotype of one or more of the capsid proteins that package the vector. In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., a AAV1, AAV3, AAV4, AAV5, AAV6, AAV7, AAV8, AAV9, AAV10, AAV11, AAV 12, RhlO, Rh74 or AAV-2i8 serotype or variant thereof.

Recombinant AAV vectors that include a polynucleotide that directs the expression of a polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction competent AAV particle is capable of binding to and entering a mammalian cell and subsequently delivering a nucleic acid cargo (e.g., a heterologous gene) to the nucleus of the cell. Thus, an intact AAV particle that is transduction-competent is configured to transduce a mammalian cell. An AAV particle configured to transduce a mammalian cell is often not replication competent and requires additional protein machinery to self-replicate. Thus, an AAV particle that is configured to transduce a mammalian cell is engineered to bind and enter a mammalian cell and deliver a nucleic acid to the cell, wherein the nucleic acid for delivery is often positioned between a pair of AAV ITRs in the AAV genome.

Suitable host cells for producing transduction competent AAV particles include but are not limited to microorganisms, yeast cells, insect cells, and mammalian cells that can be, or have been, used as recipients of a heterologous rAAV vectors. Cells from the stable human cell line 293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments and expresses the adenoviral Ela and Elb genes is used to generate recombinant AAV particles. The modified HEK293 cell line is readily transfected and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

In certain embodiments, an AAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11 , AAV 12, Rh10, Rh74 or AAV-2i8 particle. In particular embodiments, an AAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use of an AAV2 particle. In a particular aspect, an AAV2 particle is a recombinant AAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV2 particle (e.g., a capsid of an AAV2 particle) comprises a VP1 polypeptide having at least 60%, at least 70% identity, at least 75% identity, at least 80% identity, at least 85% identity, at least at least 90% identity, at least 95% identity, at least 98% identity, at least 99% identity, or even 100% identity to wild-type AAV2 VP1 capsid. In certain embodiments an AAV2 particle comprises a VP1 polypeptide that is about 63% or more identical (e.g., 63% identity) to the polypeptide having the amino acid sequence of AAV2 VP1 capsid protein. AAV2 capsid sequence and AAV4 capsid sequence are about 60% identical. In certain embodiments, the AAV2 VP1 capsid protein has a sequence that has at least 65% identity to wild-type AAV2 VP1 capsid. In certain embodiments, the AAV2 VP1 capsid protein comprises wild-type AAV2 VP1 capsid.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAVS, AAV6, AAV7, AAV8, AAV9, AAV10, AAV11, AAV 12, AAV-rh74, AAV-Rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Any suitable length of DNA can be incorporated into an AAV particle. Suitable DNA molecules for use in rAAV vectors can about 5 kilobases (kb), less than about 5 kb, less than about 4.5 kb, less than about 4 kb, less than about 3.5 kb, less than about 3 kb, or less than about 2.5 kb.

A “transgene” is used herein to conveniently refer to a nucleic acid that is intended or has been introduced into a cell or organism. Transgenes include any nucleic acid, such as a gene that encodes a polypeptide or protein (e.g., TPP1), and are generally heterologous with respect to naturally occurring AAV genomic sequences.

In a cell having a transgene, the transgene is often introduced/transferred by way of a vector, such as a rAAV particle. Introduction of a transgene into a cell by a rAAV particle is often referred to as “transduction” of the cell. The term “transduce” refers to introduction of a molecule such as a nucleic acid into a cell or host organism by way of a vector (e.g., an AAV particle). The transgene may or may not be integrated into genomic nucleic acid of a transduced cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extra chromosomally, or only transiently. A “transduced cell” is a cell into which the transgene has been introduced by way of transduction. Thus, a “transduced” cell is a cell into which, or a progeny thereof in which a nucleic acid has been introduced. A transduced cell can be propagated and the introduced protein expressed, or nucleic acid transcribed. For gene therapy uses and methods, a transduced cell can be in a mammal.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism. Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein.

Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid is “operably linked” when it is placed into a functional relationship with another nucleic acid sequence. A nucleic acid encoding a polypeptide, or a nucleic acid directing expression of a TPP1 polypeptide (e.g., a polypeptide having TPP1 activity) may include an inducible promoter, or a tissue- specific promoter for controlling transcription of the encoded polypeptide.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

### III. TPP1

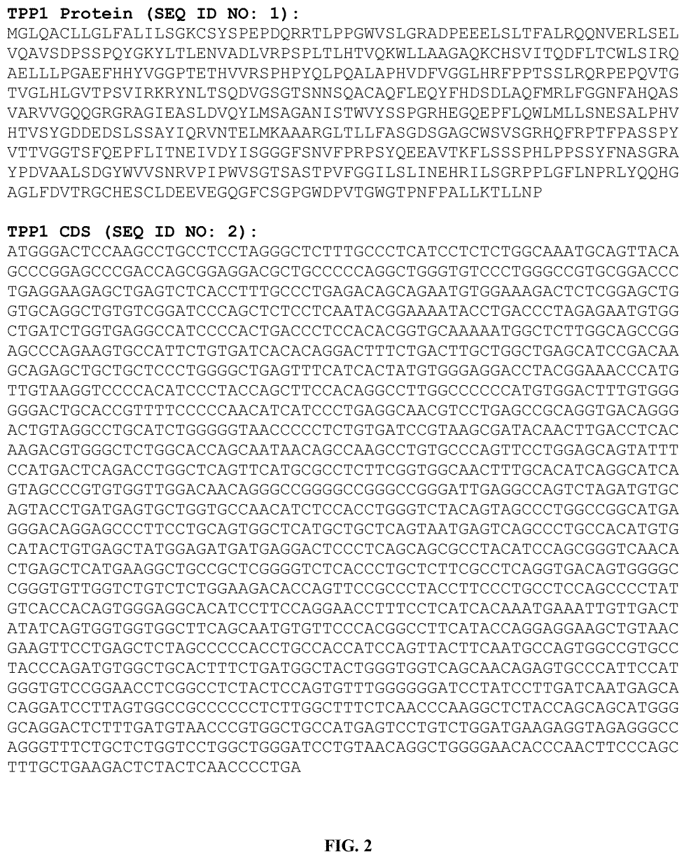

TPP1 is a lysosomal serine protease encoded by the CLN2 gene (TPP1 gene). A representative amino acid sequence for human TPP1 is set forth in FIG. 2, and a representative nucleic acid sequence for human TPP1 is set forth in FIG. 2. Human TPP1 comprises tripeptidylpeptidase I activity (TPP1 enzyme activity). TPP1 activity comprises a non-specific lysosomal peptidase activity which generates tripeptides from the breakdown products produced by lysosomal proteinases. Substrate-specificity studies indicate that TPP1 primarily cleaves tripeptides from unsubstituted amino termini in peptides and proteins. Endogenously expressed TPP1 is synthesized as a catalytically-inactive enzyme. After targeting into lysosomes, because of the acidic environment, the TPP1 is auto-catalytically processed into a mature active enzyme. The activity of TPP1 can be measured and/or quantitated in vitro using known methods. See, for example, Junaid et al. (1999).

In certain embodiments a rAAV particle comprises an AAV capsid protein and a nucleic acid encoding a polypeptide comprising TPP1 activity. In certain embodiments a rAAV particle comprises an AAV capsid protein and a nucleic acid that directs the expression and/or secretion of a polypeptide comprising TPP1 activity.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a TPP1 substitution mutant, which refers to a protein encoded by a gene having a substituted residue as compared to wild-type TPP 1.

Amino acid changes in a polypeptide can be achieved by changing the codons of the corresponding nucleic acid sequence. Such polypeptides can be obtained based on substituting certain amino acids for other amino acids in the polypeptide structure in order to modify or improve biological activity. For example, through substitution of alternative amino acids, small conformational changes may be conferred upon a polypeptide that results in increased activity. Alternatively, amino acid substitutions in certain polypeptides may be used to provide residues, which may then be linked to other molecules to provide peptide-molecule conjugates which, retain sufficient properties of the starting polypeptide to be useful for other purposes.

A polypeptide comprising TPP1 activity refers to a TPP1 protein of a mammal, or a portion thereof, that displays at least 50%, at least 60%, at least 70%, at least 75%, at least 80%, at least 85%, at least 90%, at least 95%, or about 100% of the peptidase activity of the human TPP1 as assayed using a suitable peptide substrate, for example, as assayed by the method of Junaid et al., 1999 or another comparable method. In certain embodiments a polypeptide comprising TPP1 activity refers to a TPP1 protein of a mammal, or a subsequence or variant thereof, that displays at least at least 50%, at least 60%, at least 70%, at least 75%, at least 80%, at least 85%, at least 90%, at least 95%, or about 100% of the peptidase activity of the human TPP1.

A polypeptide comprising TPP1 activity may comprise a truncated, mutated, chimeric, or modified form of a TPP1 polypeptide that retains at least partial TPP1 activity. A polypeptide comprising TPP1 activity may comprise a TPP1 protein, or a portion thereof, obtained from any suitable organism (e.g., from a mammal, from a human, from a non-human mammal, e.g., from a dog, pig, cow, or the like). In certain embodiments a polypeptide comprising TPP1 activity has at least 60% identity, at least 70% identity, at least 75% identity, at least 80% identity, at least 85% identity, at least 90% identity, at least 95% identity, at least 98% identity, or 100% identity to the human TPP1 protein.

An example of an amino acid modification is a conservative amino acid substitution or a deletion. In particular embodiments, a modified or variant sequence (e.g., TPP1) retains at least part of a function or activity of the unmodified sequence (e.g., wild-type TPP1).

One can use the hydropathic index of amino acids in conferring interactive biological function on a polypeptide, wherein it is found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated in intended for use in immunological embodiments. The greatest local average hydrophilicity of a “protein,” as governed by the hydrophilicity of its adjacent amino acids, correlates with its immunogenicity. Accordingly, it is noted that substitutions can be made based on the hydrophilicity assigned to each amino acid.

In using either the hydrophilicity index or hydropathic index, which assigns values to each amino acid, conduct substitutions of amino acids where these values are +2, with +1 being typical, and those with in +0.5 being the most typical substitutions.

Similarly, a “nucleic acid” or “polynucleotide” variant refers to a modified nucleic acid sequence which has been genetically altered compared to wild-type. The sequence may be genetically modified without altering the encoded protein sequence. Alternatively, the sequence may be genetically modified to encode a variant protein, e.g., a variant TPP1 protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a TPP1 protein encoded thereby, and some codons of the nucleic acid variant will be changed which in turn changes the amino acids of a TPP1 protein encoded thereby.

Non-limiting examples of modifications include one or more nucleotide substitutions or additions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides). One non-limiting example of a nucleic acid modification is codon optimization.

A “nucleic acid fragment” is a portion of a given nucleic acid molecule.

Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of enzymatic activity of the wild-type TPP1).

A “variant” of a TPP1 molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of enzymatic activity of the wild-type TPP1).

“Conservatively modified variations” of a particular nucleic acid sequence refers to those nucleic acid sequences that encode identical or essentially identical amino acid sequences.

Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a TPP1 polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are substantially identical is that one polypeptide is immunologically reactive with antibodies raised against the second polypeptide. Thus, a TPP1 polypeptide is substantially identical to a second TPP1 polypeptide, for example, where the two peptides differ only by a conservative substitution.

### IV. Combination Therapies

In certain embodiments a method or use includes administering or delivering AAV-TPP1 particles to a mammal and optionally administering one or more immunosuppressive agents to the mammal. In certain embodiments a method or use includes administering or delivering AAV-TPP1 particles to a mammal and optionally administering 2, 3, 4 or more immunosuppressive agents to the mammal. In certain embodiments a method or use includes administering or delivering AAV-TPP1 particles to a mammal and optionally administering two immunosuppressive agents to the mammal. In one representative embodiment, a method or use of treating a mammal includes administering or delivering AAV-TPP1 particles to a mammal and administering first and second immunosuppressive agents to the mammal.

Where two or more immunosuppressive agents are administered, each immunosuppressive agent may be distinct and/or different (e.g., each agent differs in structure and/or mechanism of action). In certain embodiments, an immunosuppressive agent is an anti-inflammatory agent. In certain embodiments, an immunosuppressive agent is mycophenolate, or a derivative thereof. An example of such a mycophenolate derivative is mycophenolate mofetil (MMF). In certain embodiments, an immunosuppressive agent is cyclosporine or a derivative thereof. In certain embodiments a first immunosuppressive agent comprises cyclosporine and a second immunosuppressive agent comprises mycophenolate, or a derivative thereof (e.g., MMF). In certain embodiments a first immunosuppressive agent comprises cyclosporine and a second immunosuppressive agent comprises MMF.

In certain embodiments, an immunosuppressive agent is administered before, during and/or after administration of AAV-TPP1 particles to a mammal. In certain embodiments, an immunosuppressive agent is administered concurrently with administration of AAV-TPP1 particles to a mammal. In certain embodiments, an immunosuppressive agent is administered after administration of AAV-TPP1 particles to a mammal.

In certain embodiments, a first immunosuppressive agent is administered to a mammal at least about 1 to about 7 days before, or about 1, about 2, about 3, about 4 or about 5 weeks before administration of AAV-TPP1 particles to a mammal and a second immunosuppressive agent is administered about 1 to about 7 days before, about 1, about 2, about 3, about 4 or about 5 weeks before, during and/or within about 10, about 20, about 30, about 40, about 50, about 100, about 200, about 300, about 350, about 400 or about 500 days after administration of AAV-TPP1 particles to the mammal. In certain embodiments, cyclosporine is administered to a mammal at least about 1 to about 7 days before, or about 1, about 2, about 3, about 4 or about 5 weeks before administration of AAV-TPP1 particles to a mammal, and mycophenolate or a derivative thereof (e.g., MMF) is administered about 1 to about 7 days before, about 1, about 2, about 3, about 4 or about 5 weeks before, during and/or within about 10, about 20, about 30, about 40, about 50, about 100, about 200, about 300, about 350, about 400 or about 500 days after administration of AAV-TPP1 particles to the mammal. In certain embodiments, cyclosporine is administered about 1 to about 7 days before, or about 1, about 2, about 3, about 4 or about 5 weeks before administration of AAV-TPP1 particles and at regular intervals after treatment, and mycophenolate or a derivative thereof (e.g., MMF) is administered once at about 1 to about 7 days before, about 1, about 2, about 3, about 4 or about 5 weeks before, during and/or within about 10 to about 40 days after administration of AAV-TPP1 particles to the mammal.

An immunosuppressive agent can be administered at any suitable dose. In certain embodiments, cyclosporine is administered at a dosage of about 1 to about 50 mg/kg, about 1 to about 20 mg/kg, or about 5 to about 10 mg/kg at a frequency of once, twice or three times a day, to once every other day. In certain embodiments cyclosporine is administered at about 10 mg/kg twice a day. In certain embodiments, cyclosporine is administered at about 10 mg/kg twice a day for a period of at least about 1, about 2, about 3, about 4 or about 5 months. In certain embodiments, a dosage of cyclosporine is tapered down to a dose of less than about 5 mg/kg, or less than about 2 mg/kg about 1 to about 2 months after administration or use of AAV-TPP1 particles to a mammal.

In certain embodiments, mycophenolate or a derivative thereof (e.g., MMF), is administered at a dosage of about 1 to about 100 mg/kg, about 1 to about 50 mg/kg, about 1 to about 25 mg/kg, or about 5 to about 20 mg/kg at a frequency of once, twice or three times a day, to once every other day. In certain embodiments, mycophenolate or a derivative thereof (e.g., MMF) is administered at about 10 to about 20 mg/kg once a day. In certain embodiments, a dosage of mycophenolate or a derivative thereof (e.g., MMF) is reduced down to a dose of less than about 5 mg/kg, or less than about 2 mg/kg about 1 to about 2 months after the administration of AAV-TPP1 particles to a mammal.

### V. Pharmaceutical Formulations, Dosages, and Routes of Administration

A rAAV particle can be formulated in any suitable formulation suitable for a subretinal administration, such as liquid formulations or lyophilized formulations that are reconstituted for use. Various pharmaceutically acceptable formulations are commercially available and obtainable by a medical practitioner.

An exemplary subretinal dosing procedure is as follows. The subject is placed in dorsal recumbency. Topical Proparacaine is applied to the eyes, the conjunctival fornices are flushed with a 1:50 dilution of betadine solution/saline, and the eyelid margins are swabbed with undiluted 5% betadine solution. A lateral canthotomy is performed using Steven's tenotomy scissors. A caliper is used to mark spots 3.0 mm posterior to the limbus on the superotemporal and inferotemporal sclera. Bipolar cautery is used to cauterize the sclera under the marked spots, followed by topical application of undiluted 5% betadine solution. Scleral fixation forceps are used to fix the globe position while a microvitreoretinal blade with a 25 gauge valved cannula is inserted at each marked spot, through the conjunctiva and sclera, and advanced into the vitreous humor. The trocar is positioned to face the posterior axis of the globe, and then retracted to leave the scleral port in place. As an optional step, a 31-gauge needle is inserted tangentially through the limbus and into the anterior chamber of the right eye to collect an aqueous humor sample (approximately 80 μL). The sample is placed on dry ice immediately post collection.

A direct contact surgical lens is placed on the cornea with sterile coupling gel and an endoilluminator probe is inserted through one of the scleral ports to facilitate direct visualization of the posterior segment through the microscope. A subretinal injection cannula is inserted through the second port and advanced into the mid-vitreous. The small diameter injection cannula is advanced until it contacted the retinal surface and placed along the inferior vascular arcade. The composition is slowly delivered to induce a subretinal bleb. If appropriate bleb formation is visualized, the injection is continued to deliver the entire dose volume into the subretinal space. If bleb formation is not visualized, the small diameter injection cannula is repositioned and the injection is attempted again at the same location or an alternative location depending on the surgeon's preference. Once the entire injection dose is delivered, the injection cannula and endoilluminator probe are removed from the scleral ports, and the contact lens is removed from the cornea. The scleral ports are removed and the lateral canthotomy site was closed using 7-0 Vicryl suture. Gentamicin and triamcinolone acetinide may be administered as a subconjunctival injection into the right eye. The procedure (administration, gentamicin/triamcinolone acetinide administration, and optionally including aqueous humor collection) is then repeated for the contralateral eye.

An immunosuppressive agent can be administered by any suitable route, such as subretinal, and accordingly formulated. In certain embodiments, an immunosuppressive agent is administered orally. In certain embodiments, mycophenolate or a derivative thereof, such as Mycophenolate Mofetil (MMF), is administered orally. In certain embodiments, cyclosporine is administered orally. An immunosuppressive agent can also be administered parenterally (e.g., intramuscularly, intravenously, subcutaneously), or administered by injection to the brain, spinal cord, or a portion thereof (e.g., injected into the CSF).

An effective amount of rAAV particles, such as AAV-TPP1 particles, can be empirically determined. Administration can be effected in one or more doses, continuously or intermittently throughout the course of treatment. Effective doses of administration can be determined by those of skill in the art and may vary according to the AAV serotype, viral titer and the weight, condition and species of mammal being treated. Single and multiple administrations can be carried out with the dose level, target and timing being selected by the treating physician. Multiple doses may be administered as is required to maintain adequate enzyme activity, for example.

In certain embodiments, a plurality of AAV-TPP1 particles are administered. As used herein, a plurality of AAV particles refers to about 1×10⁵ to about 1×10¹⁶ particles.

In certain embodiments, rAAV particles, such as AAV-TPP1 particles, are administered at a dose of about 1×10⁵ to about 1×10¹⁶ vg/ml in about 500 μl to about 5 ml; at a dose of about 500 μl to about 3 ml of 1×10⁵ to about 1×10¹⁶ vg/ml; or at a dose of about 500 μl to about 2 ml of 1×10⁵ to about 1×10¹⁶ vg/ml. In certain embodiments, rAAV particles, such as AAV-TPP1 particles, are administered at a dose of about 1×10⁸ to about 1×10¹⁶ vg/kg body weight of the mammal being treated. For example, rAAV particles, such as AAV-TPP1 particles, can be administered at a dose of about 1×10⁸ vg/kg, about 5×10⁸ vg/kg, about 1×10⁹ vg/kg, about 5×10⁹ vg/kg, about 1×10¹⁰ vg/kg, about 5×10¹⁰ vg/kg, about 1×10¹¹ vg/kg, about 5×10¹¹ vg/kg, about 1×10¹² vg/kg, about 5×10¹² vg/kg, about 1×10¹³ vg/kg, about 5×10¹³ vg/kg, about 1×10¹⁴ vg/kg, about 5×10¹⁴ vg/kg, or about 1×10¹⁵ vg/kg body weight of the mammal being treated. Specific sub-retinal dosages include 7.5×10¹¹ vg/eye, 2.5×10¹¹ vg/eye, 8.3×10¹⁰ vg/eye, 8.3×10⁹ vg/eye, and 5.0×10⁹ vg/eye, or 1×10⁸ to 1×10¹² per eye.

Pharmaceutical forms suitable for injection or infusion of rAAV particles, such as AAV-TPP1 particles, can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of rAAV particles, such as AAV-TPP1 particles, can optionally include the following components: a sterile diluent such as water for injection, saline solution, such as phosphate buffered saline (PBS), artificial CSF, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

rAAV particles, such as AAV-TPP1 particles, may be provided as a lyophilized composition. In certain embodiments, the formulation is lyophilized from a liquid formulation. In some aspects, the liquid formulation comprises about 5 mM to about 25 mM, about 5 mM to about 15 mM, about 10 mM to about 20 mM, or about 15 mM to about 25 mM of a buffering agent. In exemplary aspects, the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM of a buffering agent. Pharmaceutically acceptable buffering agents are well known in the art, and include without limitation, phosphate buffers, histidine, sodium citrate, HEPES, Tris, Bicine, glycine, N-glycylglycine, sodium acetate, sodium carbonate, glycyl glycine, lysine, arginine, sodium phosphate, and mixtures thereof. In certain embodiments, the buffer is histidine (e.g., L-histidine).

Inclusion of moderate levels (i.e., between about 1% to about 10%) of one or more sugar and/or sugar alcohol assists in the stability of the liquid and/or lyophilized formulations. For example, the sugar and/or sugar alcohol allows for better properties during freeze/thawing cycles. Accordingly, in certain embodiments, the present invention provides pharmaceutical compositions containing between about 2% and about 10% of one or more sugars and/or sugar alcohols. Any sugar such as mono-, di-, or polysaccharides, or water-soluble glucans, including for example fructose, glucose, mannose, sorbose, xylose, maltose, lactose, sucrose, dextran, trehalose, pullulan, dextrin, cyclodextrin, soluble starch, hydroxyethyl starch, and carboxymethylcellulose may be used. In a particular embodiment, the sugar is sucrose, trehalose, or a combination thereof. In certain embodiments, the trehalose is trehalose dihydrate. Sugar alcohols are defined as a hydrocarbon having between about 4 and about 8 carbon atoms and a hydroxyl group. Non-limiting examples of sugar alcohols that may be used in the pharmaceutical compositions provided herein include, mannitol, sorbitol, inositol, galactitol, dulcitol, xylitol, and arabitol. In certain embodiments, mannitol is used as a sugar alcohol additive. In certain embodiments, a pharmaceutical composition contains both a sugar and a sugar alcohol additive. [0061] The sugars and sugar alcohols may be used individually or in combination. In some embodiments, the sugar, sugar alcohol, or combination thereof will be present in the formulation at a concentration of about 1% to about 10% (w/v), about 1% (w/v) to about 1.5% (w/v), about 2.5% to about 7.5% (w/v), or about 1% to about 5% (w/v). In exemplary aspects, the pharmaceutical composition of the present disclosure comprises about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), about 2.5% (w/v), about 3.0% (w/v), about 3.5% (w/v), about 4.0% (w/v), about 4.5% (w/v), about 5.0% (w/v), about 5.5% (w/v), about 6.0% (w/v), about 6.5% (w/v), about 7.0% (w/v), about 7.5% (w/v), about 8.0% (w/v), about 8.5% (w/v), about 9.0% (w/v), about 9.5% (w/v), or about 10% (w/v) sugar, sugar alcohol, or combination thereof. In certain embodiments, the sugar is sucrose, trehalose, or a combination thereof. In certain embodiments, the trehalose is trehalose dihydrate.

In exemplary embodiments, the formulations or pharmaceutical compositions of the present disclosure comprise additional pharmaceutically acceptable ingredients. In exemplary aspects, the formulations or pharmaceutical compositions comprise any one or a combination of the following: acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

A lyophilized composition may be reconstituted with water or buffer/buffering agent to produce a reconstituted dosage unit suitable for administration to a subject. In certain embodiments, the reconstituted dosage unit has a pH compatible with physiological conditions. In some cases, the pH of the reconstituted dosage unit ranges from 6 to 8. In some cases, the pH of the reconstituted dosage unit ranges from 7 to 8. For example, the pH of the reconstituted dosage unit may range from 7 to 7.5.

rAAV particles, such as AAV-TPP1 particles, may be formulated with a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenyhnercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the pharmaceutical composition to ensure protection against secondary contaminations during use caused by bacteria and fungi. In another embodiment, the pharmaceutical formulations of this invention do not include a preservative. The concentration of the preservative component, if any, in the present compositions is a concentration effective to preserve the composition, and is often in a range of about 0.00001% to about 0.05% or about 0.1% (w/v) of the composition, and so forth to include all values within the range including the endpoints of the range where appropriate.

rAAV particles, such as AAV-TPP1 particles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of rAAV particles (e.g., AAV-TPP1 particles) believed necessary to produce the desired effect(s). The amount necessary can be formulated in a single dose or can be formulated in multiple dosage units. The dose may be adjusted to a suitable rAAV particles concentration, optionally combined with an anti-inflammatory agent, and packaged for use.

In one embodiment, pharmaceutical compositions will include sufficient genetic material (rAAV particles) to provide a therapeutically effective amount, i.e., an amount sufficient to reduce or ameliorate symptoms of a disease state in question or an amount sufficient to confer the desired benefit. Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween⁸⁰, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences, 1991.

Formulations containing rAAV particles, such as AAV-TPP1 particles, will contain an effective amount of the rAAV particles in a vehicle, the effective amount being readily determined by one skilled in the art. The rAAV particles, such as AAV-TPP1 particles, may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

In certain embodiments a method includes administering a plurality of rAAV particles, such as AAV-TPP1 particles, to a mammal (e.g., a mammal having an LSD such as LINCL) as set forth herein, where severity, frequency, progression or time of onset of one or more symptoms of a LSD are decreased, reduced, prevented, inhibited or delayed. In some cases, the symptoms of a LSD are decreased for 1, 2, 3, 4, 5, 6, 7, 8, 9, 10, 11, 12, 13 or 14 days, 1, 2, 3, 4, 5, 6, 7, or 8 weeks, or 1, 2, 3, 4, 5, or 6 months. As such, in some cases, a treatment regimen includes, but is not necessarily limited to, once per day, three times per week, twice per week, once per week, once every two weeks, once every three weeks, once per month, once every 5 weeks, once every 6 weeks, once every 7 weeks, once every other month, and any combination thereof. The term “time of onset” refers to a point in time after a first administration of AAV-TPP1 particles that a symptom of LSD is first observed or detected. Non-limiting symptoms of LSD in which severity, frequency, progression or time of onset of one or more symptoms of a LSD are decreased, reduced, prevented, inhibited or delayed include a proprioceptive response, nystagmus, menace, pupillary light reflex, cerebellar ataxia and intention tremor. The severity, frequency, progression or time of onset of one or more symptoms of a LSD can be subjectively determined by a standardized clinical neurologic examination (e.g., see Lorenz et al., 2011).

A delay in the time of onset of a symptom associated with LSD can be determined by comparing the time of onset of a symptom for a mammal treated with AAV-TPP1 particles to one or more mammals treated without AAV-TPP1 particles. In certain embodiments a method includes administering a plurality of AAV-TPP1 particles to the central nervous system, or portion thereof, of a mammal (e.g., a mammal having an LSD) and severity, frequency, progression or time of onset of one or more symptoms of a LSD are decreased, reduced, prevented, inhibited or delayed by at least about 5 to about 10, about 10 to about 25, about 25 to about 50, or about 50 to about 100 days.

### VI. Definitions

The terms “polynucleotide” and “nucleic acid” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an “open reading frame” or ORF that encodes the polypeptide. Unless otherwise indicated, a particular nucleic acid sequence also includes degenerate codon substitutions.

The term “polypeptide” as used herein refers to a polymer of amino acids and includes full-length proteins and fragments thereof. Thus, “protein”, “polypeptide”, and “peptide” may often be used interchangeably herein. The “polypeptides” encoded by a “nucleic acid” or “polynucleotide” sequence disclosed herein include partial or full-length native TPP1 sequences, as with naturally occurring wild-type and functional polymorphic proteins, functional subsequences (fragments) thereof, and modified forms or sequence variants thereof, so long as the polypeptide retains some degree of TPP1 enzyme activity. Accordingly, in methods of the invention, such polypeptides encoded by nucleic acid sequences can be, but are not required to be, identical to the endogenous protein TPP1 protein that is defective, or whose expression is insufficient, or deficient in a treated mammal.

The term “vector” refers to small carrier nucleic acid molecule, a plasmid, virus (e.g., AAV vector), or other vehicle that can be manipulated by insertion or incorporation of a nucleic acid. Vectors such as AAV can be used to introduce/transfer polynucleotides into cells, such that the polynucleotide therein is transcribed and subsequently translated by the cells.

An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), polyadenylation signal.

A polypeptide can be targeted for delivery to an extracellular, intracellular or membrane location. A gene product secreted from cells typically has a secretion “signal” for secretion from the cell to the extracellular milieu. An expression vector can also be constructed to include a secretion “signal.” A gene product may also be retained within the cell. In a similar manner, a gene product may include, or the expression vector can be constructed to include, “retention” signal sequences for anchoring the polypeptide within the cell plasma membrane. For example, membrane proteins have hydrophobic transmembrane regions, which maintain protein in the membrane.

The term “about” at used herein refers to a values that are within 10% (plus or minus) of a reference value.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of one or more symptoms, diminishment of extent of disease, stabilizing (i.e., not worsening or progressing) one or more symptoms or state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The terms “a” and “an” and “the” and similar referents in the context of describing the invention are to be construed to cover both the singular and the plural, unless otherwise indicated herein or clearly contradicted by context. Thus, for example, reference to “a vector” includes a plurality of such vectors, reference to “a virus” or “particle” includes a plurality of such virions/particles and reference to “AAV or rAAV particle” includes a plurality of such AAV or rAAV particles.

The terms “comprising,” “having,” “including,” and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein.

All numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth to include all values within the range including the endpoints of the range where appropriate.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth to include all values within the range including the endpoints of the range where appropriate.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc. and all values within the range including the endpoints of the range where appropriate

### VII. EXAMPLES

The disclosure includes all modifications and equivalents of the subject matter recited in the claims appended hereto as permitted by applicable law. Moreover, any combination of the elements in all possible variations thereof is encompassed by the disclosure unless otherwise indicated herein or otherwise clearly contradicted by context. Accordingly, the following examples are intended to illustrate but not limit the scope of the invention claimed.

### Example 1—Cynomolgus Monkey Studies

The objective of this study was to evaluate transgene expression following subretinal injection of the test article AAV-TPP1 (7.5×10¹¹, 2.5×10¹¹, or 8.3×10¹⁰ vg/eye) in monkeys. Ophthalmoscopic examinations indicated RPE/choroid pigmentation was somewhat altered at all three dose levels during the study and OCT image analysis revealed alterations in retinal morphology. There were no test article-related effects on hematology or clinical chemistry endpoints at any dose level. Given that the first study showed some damage as well as expression levels that were well above that needed for therapeutic benefit, a second cynomolgus monkey study was done. In study 2, transgene expression was assessed following subretinal injection of AAV-TPP1 at 5×10⁹ vg/eye and 8.3×10⁹ vg/eye. Both doses were well tolerated in the non-human primates.

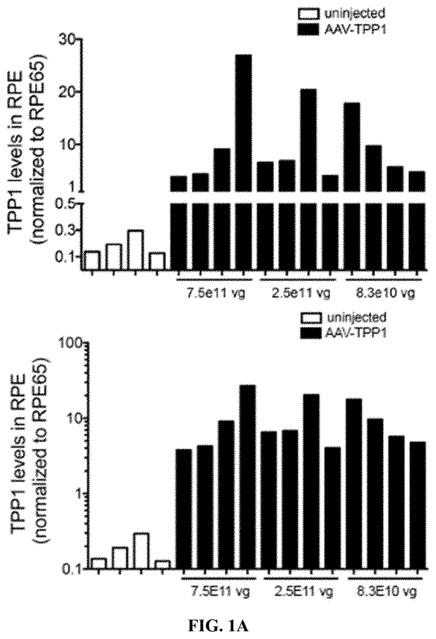

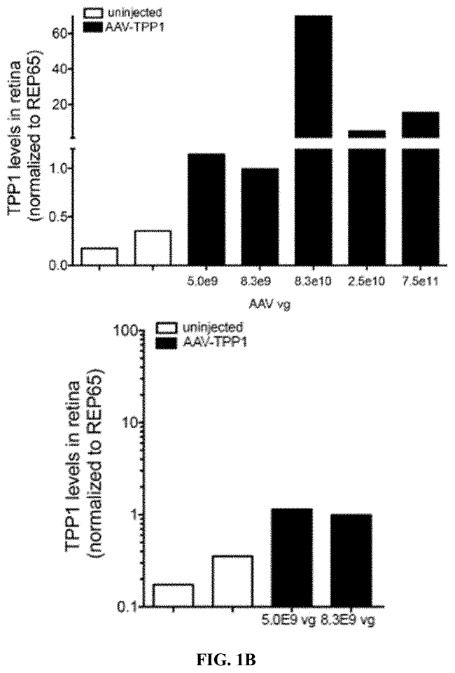

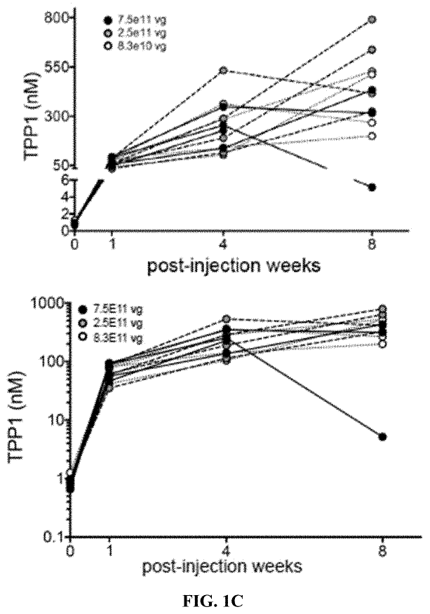

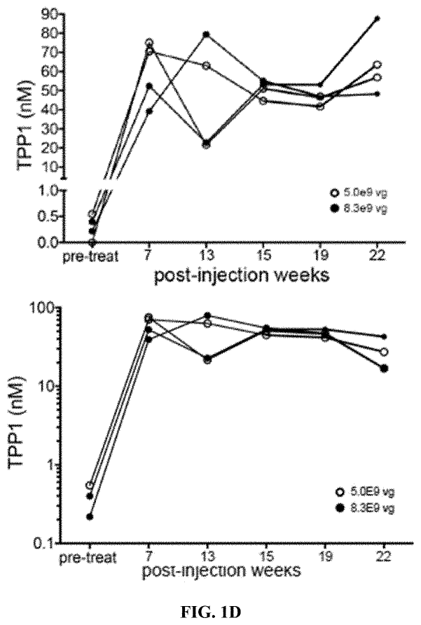

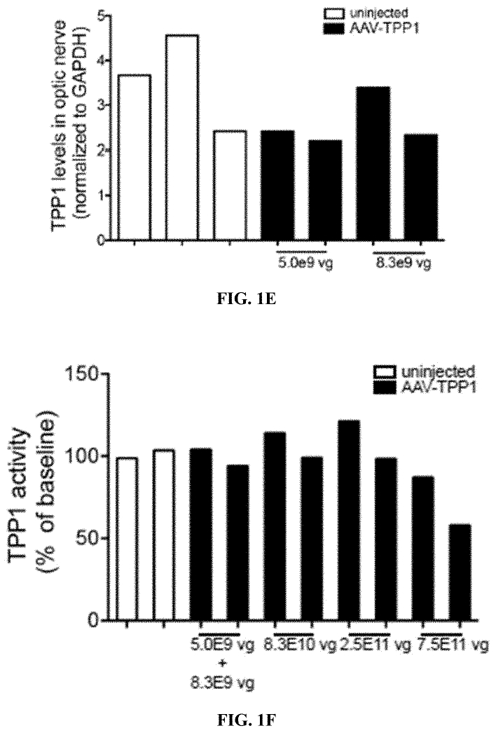

TPP1 levels were measured following all doses in the aqueous humor at various timepoints (FIGS. 1C-D) and in the retina and optic nerve following necropsy. (FIGS. 1A, FIG. 1B and FIG. 1E). The percentage of TPP1 activity was determined after incubation of TPP1-deficient cells with recombinant TPP1 and serum from control or injected animals (FIG. 1F). Decreased TPP1 activity can be quantified in samples from animals which developed neutralizing antibodies against the recombinant TPP 1.

The inventors know that achieving levels of 10-time higher than endogenous levels of hTPP1 is efficacious in animal models (Katz et al. Sci Trans. Med. 2015). Currently, they can achieve 100-time endogenous levels at 5×10⁹ vg/eye. Extrapolating from this, the dose can be safely reduced by one log and still reach an efficacious dose with even less viral load.

### Example 2—Human Clinical Trial Protocol

An open-label, non-randomized, dose-escalation study to evaluate the safety, tolerability, and efficacy of sub-retinal infusion of AAV2-TPP1 in subjects with TPP1 deficiency and receiving ERT is proposed. The proposed study will evaluate bilateral injections (minimum of 3 weeks between surgeries) of AAV2-TPP1 at two doses (may only be one dose if lower dose proves efficacious).

In this dose escalation study, initial subjects will receive the starting dose of TBD vg/eye and complete at least six (6) months of safety observation prior to dosing the first subject at the next dose-level. A second cohort will receive the higher dose level of TBD vg/eye (or the lower dose if proven safe and efficacious after six months), a minimum of six months after the initial subjects. If pre-specified TPP1 levels are reached at the low dose, the trial will continue with all subjects at this dose.

All injected subjects will undergo safety observation for a total of 52 (±2) weeks after AAV delivery to the eye. Subjects who complete 52 (±2) weeks (End-of-Study) will be encouraged to enroll in an extension study evaluating the long-term safety of AAV2-TPP1 for an additional 4 years.

At least 2 weeks will lapse between administrations within a dose cohort to ensure safety. Subjects will undergo regularly scheduled ERT at their normally used sites for infusions. Five to six days post-ERT, travel to study site to have the first eye surgery/gene therapy infusion. Patients to return to study site in a minimum of 3 weeks for second eye surgery.

The primary efficacy endpoint is reduction in retinal disease (both visual and measurable) including Morphology OCT and functionality ERG or pupillometry:
Quantification of subfields of the macula—Increase in diameter of hyper auto fluorescent ring around the atrophic retina-ring can be an outcome measure of where you stop the disease or slow it down; use the blue light plus the OCT to determine increase in ring diameter (reminder min. of 6 months post gene therapy infusion prior to being able to assess
Vector shedding analysis of AAV2-TPP1

Pharmacokinetic parameter measurements of vector-derived hTTP1 enzymatic activity levels in aqueous humor fluid and total protein levels will be obtained if possible.

The secondary efficacy endpoints may be measured by:
Pupillary light reflex testing
Full-field light sensitivity threshold testing
Visual Acuity

The exploratory efficacy endpoint may be measured by:
Visual and retinal function will also be measured using:

a. Visual function questionnaire

b. Visual field testing—Humphrey and/or Goldmann

c. Contrast sensitivity
Health-related quality-of-life by CHQ (i.e., CHQ-PF-28)

To be eligible to participate in this study, candidates must meet the following eligibility criteria at screening:
Classic late infantile: first seizure by age of 4 in patient or older sibling (if patient still younger) OR two common mutations with language delay (4 or younger), AND total score of ≥3.
Have confirmed diagnosis of TPP1 deficiency (based on both genotype and hTPP1 enzymatic activity).
No previous participation in a gene therapy study for TPP1 deficiency (except ERT for TTP1 deficiency).
Subjects undergoing ERT minimum of 6 months to a maximum of 2 years.
Have documented total score of ≥3 in the motor and language domains of a total 6-point rating scale.

Also, parents or legal guardians and subject, if appropriate, must able to understand the purpose and risks of the study and provide signed and dated informed consent and authorization to use protected health information (PHI) for their child's participation in accordance with national and local privacy regulations.

Candidates will be excluded from study entry if any of the following exclusion criteria exist at Screening:
Unable or unwilling to meet requirements of the study, including receiving bilateral subretinal vector administrations.
Not currently receiving ERT for TTP1 deficiency.
Any prior participation in a study in which a gene therapy vector was administered.
Participation in a clinical study with an investigational drug in the past six months.
Use of compounds or precursors that could potentially interact with the biochemical activity of the TPP1 enzyme; individuals who discontinue use of these compounds for 3 months may become eligible.
Prior intraocular surgery within six months.
Known sensitivity to medications planned for use in the peri-operative period.
Pre-existing eye conditions or complicating systemic diseases that would preclude the planned surgery or interfere with the interpretation of study.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

All references, patents, patent applications, articles, and literature cited in this application are specifically incorporated herein by reference. The following references may provide exemplary procedural or other details supplementary to those set forth herein.

Junaid et al., “A novel assay for lysosomal pepstatin-insensitive proteinase and its application for the diagnosis of late-infantile neuronal ceroid lipofuscinosis,” Clin. Chim. Acta., 281(1-2):169-176, 1999.

Katz et al., “AAV gene transfer delays disease onset in a TPP1-deficient canine model of the late infantile form of Batten disease,” Sci. Transl. Med., 7:313ra180, 2015.

Lorenz et al., “Handbook of veterinary neurology,” St. Louis: Elsevier Saunders, 2011.

Sambrook et al., “Molecular Cloning, a Laboratory Manual,” Cold Spring Harbor Laboratories, New York, 1989.

Wright, “Manufacturing and characterizing AAV-based vectors for use in clinical studies,” Gene Therapy, 15(11):840-848, 2008.

Wright, “Transient transfection methods for clinical adeno-associated viral vector production,” Human Gene Therapy, 20(7):698-706, 2009.

1. A method of treating a mammal having a lysosomal storage disease (LSD) that causes vision loss, said method comprising sub-retinal administering to a mammal in need thereof a plurality of AAV particles, said AAV particles comprising (i) a nucleic acid inserted between a pair of AAV inverted terminal repeats (ITRs), said nucleic acid encoding (1) a soluble lysosomal tripeptidyl peptidase 1 (TPP1) polypeptide, (2) a fragment thereof, (3) a proenzyme of a TPP1 polypeptide or a fragment thereof, or (4) a combination of any of the foregoing; and (ii) an expression control element operably linked to and driving expression of said nucleic acid to yield a polypeptide having lysosomal hydrolase activity, wherein said AAV particles are capable of transducing cells of said mammal and providing expression of said polypeptide.

2. The method of claim 1, wherein one or more of the AAV ITRs comprise one or more AAV2 ITRs.

3. The method of claim 1, wherein the nucleic acid encodes mammalian TPP1 or a biologically functional fragment thereof.

4. The method of claim 1, wherein the nucleic acid encodes human TPP1 or a biologically functional fragment thereof.

5. The method of claim 1, wherein the method results in a slowing, stopping, reversing or preventing of vision loss/blindness.

6. The method of claim 1, wherein the expression control element comprises a CMV enhancer.

7. The method of claim 1, wherein the expression control element comprises a beta actin promoter.

8. The method of claim 1, wherein the expression control element comprises a chicken beta actin promoter.

9. The method of claim 1, wherein the expression control element comprises a CMV enhancer and a chicken beta actin promoter.

10. The method of claim 1, wherein the expression control element comprises a sequence having about 80% or more identity to a native CMV enhancer or about 80% or more identity to a native chicken beta actin promoter.

11. The method of claim 1, wherein the AAV particles further comprise an AAV capsid protein or a functional fragment thereof.

12. The method of claim 11, wherein the capsid sequence comprises a VP1, VP2 and/or VP3 capsid sequence having about 70% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences.

13. The method of claim 11, wherein the capsid sequence comprises a VP1 capsid sequence having about 80% or more identity to AAV2, wherein the capsid sequence has a tyrosine at positions 444, 500 and/or 730 substituted with an amino acid that is not tyrosine.

14. The method of claim 11, wherein the capsid sequence comprises a VP1 capsid sequence having about 90% or more identity to AAV2, wherein the capsid sequence has a tyrosine at positions 444, 500 and/or 730 substituted with phenylalanine.

15. The method of claim 11, wherein the capsid sequence comprises an AAV2 VP1 capsid sequence having a tyrosine at positions 444, 500 and/or 730 substituted with phenylalanine.

16. The method of claim 11, wherein the capsid sequence comprises a VP1, VP2 or VP3 capsid sequence selected from any of: AAV1, AAV2, AAV3, AAV4, AAV5, AAV6, AAV7, AAV8, AAV9, AAV10, AAV11, Rh10, Rh74 or AAV-2i8 AAV serotypes.

17. The method of claim 1, wherein said patient previously, currently or will receive TPP1 enzyme replacement therapy through a distinct route of administration.

18. The method of claim 1, wherein said AAV particles are administered at a dose of about 1×10⁸ to about 1×10¹² total vg.

19. The method of claim 1, wherein said mammal is a non-rodent mammal.

20. The method of claim 19, wherein said non-rodent mammal is a primate.

21. The method of claim 19, wherein said non-rodent mammal is a human.

22. The method of claim 21, wherein said human is a child.

23. The method of claim 22, wherein said child is from about 1 to about 4 years of age.

24. The method of claim 1, wherein said LSD is infantile or late infantile ceroid lipofuscinoses (LINCL), Juvenile Batten, Fabry, MLD, Sanfilippo A, Krabbe, Morquio, Niemann-Pick C, Tay-Sachs, Hurler (MPS-I H), Sanfilippo B, Maroteaux-Lamy, Niemann-Pick A, Cystinosis, Hurler-Scheie (MPS-I H/S), Sly Syndrome (MPS VII), Scheie (MPS-I S), Infantile Batten, GM1 Gangliosidosis, Mucolipidosis type II7ΠI, or Sandhoff disease.

25. The method of claim 1, wherein onset of a symptom associated with said LSD is delayed by about 5-about 10, about 10-about 25, about 25-about 50, or about 50-about 100 days.

26. The method of claim 25, wherein said symptom is selected from the group consisting of proionceptive response, nystagmus, menace, pupillary light reflex, cerebellar ataxia and intention tremor.

27. The method of claim 25, wherein said symptom is vision loss or blindness.

28. The method of claim 1, wherein said AAV particles are selected from the group consisting of AAV1, AAV2, AAV3, AAV4, AAVS, AAV6, AAV7, AAV8, AAV9, AAV10, AAV11, AAV 12, AAV-rh74, AAV-Rh10 and AAV-2i8 particles.

29. The method of claim 1, wherein one or more of said ITRs is selected from the group consisting of an AAV1, AAV2, AAV3, AAV4, AAV5, AAV6, AAV7, AAV8, AAV9, AAV10, AAV11, AAV 12, AAV-rh74, AAV-Rh10 and AAV-2i8 ITR.

30. The method of claim 11, wherein the capsid sequence comprises a VP1, VP2 and/or VP3 capsid sequence having about 90% or more identity to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences.

31. (canceled)
